(19) United States
(12) Patent Application Publication
(10) Pub. No.: US 2010/0292537 A1
(43) Pub. Date: Nov. 18, 2010

(54) INNER PRESSURE DETECTION DEVICE AND INNER PRESSURE DETECTION METHOD FOR INFLATION AND DEFLATION MEMBER, AND ENDOSCOPE APPARATUS

(76) Inventors: Tsuyoshi Ashida, Ashigarakami-gun (JP); Takayuki Nakamura, Ashigarakami-gun (JP); Shinichi Yamakawa, Ashigarakami-gun (JP)

Correspondence Address: BIRCH STEWART KOLASCH & BIRCH, PO BOX 747, FALLS CHURCH, VA, 22040-0747, US

(21) Appl. No.: 12/779,251

(22) Filed: May 13, 2010

(30) Foreign Application Priority Data
May 14, 2009 (JP) 2009-117657

(51) Int. Cl. A61B 1/00; G01L 7/00; G01F 1/00

(52) U.S. Cl. 600/116; 702/50; 702/45

(57) ABSTRACT

An inner pressure detection device for an inflation and deflation member includes: a pressure detection device which detects a pressure of a fluid at a first position in a proximal end side of a tube for supplying a fluid to an inflation and deflation member and exhausting a fluid from the inflation and deflation member, the inflation and deflation member included in an insertion portion configured to be inserted in a body cavity; a pressure loss calculation device which calculates a pressure loss of the fluid in the tube between the first position and a position in a distal end side of the tube where the inflation and deflation member is provided; and an inner pressure calculation device which calculates an inner pressure of the inflation and deflation member based on the pressure loss calculated by the pressure loss calculation device and the pressure detected by the pressure detection device.

### CROSS-REFERENCE TO RELATED APPLICATIONS

This application claims the priority benefit under 35 U.S.C. §119 of Japanese Patent Application No. 2009-117657 filed on May 14, 2009, which is hereby incorporated in its entirety by reference.

### BACKGROUND OF THE INVENTION

1. Field of the Invention

The presently disclosed subject matter relates to an inner pressure detection device and an inner pressure detection method for an inflation and deflation member, and an endoscope apparatus, and particularly relates to an inner pressure detection device and an inner pressure detection method for an inflation and deflation member included in an insertion portion configured to be inserted in a body cavity such as an endoscope, a supplementary tool for an endoscope or a treatment tool for an endoscope, and an endoscope apparatus including the inner pressure detection device.

2. Description of the Related Art

Japanese Patent Application Laid-Open No. 2002-301019 discloses detecting the inner pressure of a balloon provided at a distal end portion of an endoscope or a supplementary tool for an endoscope by attaching a pressure sensor in the vicinity of a pump via a supply/exhaust tube for supplying and exhausting the gas in the balloon.

### SUMMARY OF THE INVENTION

However, according to Japanese Patent Application Laid-Open No. 2002-301019, due to a pressure loss of the supply/exhaust tube depending on a flow velocity of the gas when supplying and exhausting the gas, it is difficult to detect the inner pressure of the balloon accurately. More specifically, when the gas is supplied to the balloon, the pressure value measured by the pressure sensor can be calculated by adding the pressure loss to the true inner pressure of the balloon. Then, the pressure at the position of the pressure sensor can be expressed in the following equation:

(Pressure at a position of a pressure sensor)=(Inner pressure of a balloon)+(Pressure loss of supply/exhaust tube).

Further, when the gas is exhausted from the balloon, the pressure value measured by the pressure sensor can be calculated by subtracting the pressure loss from the true inner pressure of the balloon. Then, the pressure at the position of the pressure sensor can be expressed in the following equation:

(Pressure at a position of a pressure sensor)=(Inner pressure of a balloon)−(Pressure loss of the supply/exhaust tube).

Therefore, when supplying the gas in order to increase the inner pressure of the balloon to a predetermined pressure P0, the pump can be controlled so as to increase the pressure value measured by the pressure sensor to the predetermined pressure P0. Since the pressure value measured by the pressure sensor can be indicated by (the inner pressure of the balloon)+(the pressure loss of the supply/exhaust tube), the inner pressure of the balloon cannot reach the predetermined pressure P0. Accordingly, the operation of the pump can be suppressed, and the performance of the pump can come down. As a result, much time can be required for inflating the balloon.

Further, when exhausting the gas from the balloon, the performance of the pump can come down due to the pressure loss of the supply/exhaust tube. As a result, much time can be required for deflating the balloon.

In order to solve the aforementioned problems, a pressure sensor can be attached inside the balloon or in the vicinity of the balloon. However, the solution is not desirable since the distal end portion of an endoscope or a supplementary tool for an endoscope can increase in size.

The presently disclosed subject matter is made in view of the above circumstances, and has an object to provide an inner pressure detection device and an inner pressure detection method for an inflation and deflation member, and an endoscope apparatus, which can accurately detect an inner pressure of the inflation and deflation member such as a balloon which is provided at an insertion portion configured to be inserted in a body cavity such as an endoscope, a supplementary tool for an endoscope or a treatment tool for an endoscope.

In order to attain the above-described object, the first aspect of the presently disclosed subject matter provides an inner pressure detection device for an inflation and deflation member, including: a pressure detection device which detects a pressure of a fluid at a first position in a proximal end side of a tube for supplying a fluid to an inflation and deflation member and exhausting a fluid from the inflation and deflation member, the inflation and deflation member included in an insertion portion configured to be inserted in a body cavity; a pressure loss calculation device which calculates a pressure loss of the fluid in the tube between the first position and a position in a distal end side of the tube where the inflation and deflation member is provided; and an inner pressure calculation device which calculates an inner pressure of the inflation and deflation member based on the pressure loss calculated by the pressure loss calculation device and the pressure detected by the pressure detection device.

According to the presently disclosed subject matter, the inner pressure of the inflation and deflation member provided at the intracavital insertion portion can be accurately detected since the pressure loss is taken into the calculation of the inner pressure.

The second aspect of the presently disclosed subject matter provides an inner pressure detection device according to the first aspect, further including: a flow rate detection device which detects a flow rate of the fluid in the tube, wherein the pressure loss calculation device calculates the pressure loss based on the flow rate detected by the flow rate detection device.

The third aspect of the presently disclosed subject matter provides an inner pressure detection device according to the second aspect, wherein the pressure loss calculation device calculates the pressure loss by using a table defining a relationship of the flow rate and the pressure loss.

The fourth aspect of the presently disclosed subject matter provides an inner pressure detection device according to the second or third aspect, wherein the pressure detection device is provided in a vicinity of a supply and exhaust device which supplies and exhausts the fluid in the tube, and the flow rate detection device detects the flow rate based on the inner pressure detected by the pressure detection device by using information of a pressure flow rate characteristic of the supply and exhaust device, the flow rate characteristic defining a relationship of a drive control parameter of the supply and exhaust device, a pressure of the fluid supplied and exhausted by the supply and exhaust device and the flow rate of the fluid.

The fifth aspect of the presently disclosed subject matter provides an inner pressure detection device according to the first aspect, further including: a pressure difference detection device which detects a pressure difference of the fluid between the first position and a second position in the tube, wherein the pressure loss calculation device calculates the pressure loss based on the pressure difference detected by the pressure difference detection device.

The sixth aspect of the presently disclosed subject matter provides an inner pressure detection device according to the first aspect, further including: a pressure difference calculation device, wherein the pressure detection device detects pressure values at the first position and a second position in the tube, the pressure difference calculation device calculates a pressure difference of the fluid between the first position and the second position based on the pressures detected by the pressure detection device, and the pressure loss calculation device calculates the pressure loss based on the pressure difference calculated by the pressure difference calculation device.

The seventh aspect of the presently disclosed subject matter provides an inner pressure detection device according to the fifth or sixth aspect, wherein the pressure loss calculation device calculates a pressure loss PL1 by using an expression defined as PL1=PL2×(L1/L2) when a length of the tube between the first position and a position where the inflation and deflation member is provided is set as L1, a length of the tube between the first position and the second position is set as L2, the pressure loss is set as PL1, and the pressure difference of the fluid between the first position and the second position is set as PL2.

The eighth aspect of the presently disclosed subject matter provides an inner pressure detection device according to the first to seventh aspect, wherein the insertion portion is an insertion portion included in an endoscope.

The ninth aspect of the presently disclosed subject matter provides an inner pressure detection device according to the first to seventh aspect, wherein the insertion portion is a supplementary tool for an endoscope through which an insertion portion included in an endoscope is inserted.

The tenth aspect of the presently disclosed subject matter provides an inner pressure detection device according to the first to seventh aspect, wherein the insertion portion is an endoscope treatment tool which is led out from a forceps port of an endoscope.

In order to attain the above described object, the eleventh aspect of the presently disclosed subject matter provides an endoscope apparatus including: an insertion portion configured to be inserted into a body cavity; an inflation and deflation member provided at the insertion portion; a tube through which a fluid is supplied to and exhausted from an inside of the inflation and deflation member; a supply and exhaust device which supplies and exhausts the fluid in the tube; a pressure detection device which detects a pressure of the fluid at a first position in a proximal end side of the tube; a pressure loss calculation device which calculates a pressure loss of the fluid in the tube between the first position and a position in a distal end side of the tube where the inflation and deflation member is provided; and an inner pressure calculation device which calculates an inner pressure of the inflation and deflation member based on the pressure loss calculated by the pressure loss calculation device and the pressure detected by the pressure detection device.

According to the presently disclosed subject matter, the inner pressure of the inflation and deflation member can be accurately detected. Therefore, the supply and exhaust device can be operated with the maximum capacity to supply or exhaust the fluid of the inside of the inflation and deflation member, and inflation or deflation of the inflation and deflation member can be quickly performed.

In order to attain the above described object, the twelfth aspect of the presently disclosed subject matter provides an inner pressure detection method for an inflation and deflation member, including the step of: detecting a pressure of a fluid at a first position in a proximal end side of a tube through which a fluid is supplied to and exhausted from an inside of an inflation and deflation member included in an insertion portion; calculating a pressure loss of the fluid in the tube between the first position and a position in a distal end side of the tube where the inflation and deflation member is provided; and calculating an inner pressure of the inflation and deflation member based on the calculated pressure loss and the detected pressure at the first position.

According to the presently disclosed subject matter, the inner pressure of the inflation and deflation member such as a balloon which is provided at an insertion portion such as an endoscope, a supplementary tool for an endoscope or a treatment tool for an endoscope can be accurately detected.

### BRIEF DESCRIPTION OF THE DRAWINGS

FIG. 1 is a configuration diagram illustrating an embodiment of an endoscope apparatus;

FIG. 2 is an enlarged perspective view of a distal end portion;

FIG. 3 is a sectional view of a distal end of an insertion portion and vicinity thereof;

FIG. 4 is a schematic configuration diagram of a balloon control device according to the first embodiment;

FIG. 5 is a flowchart illustrating a method for detecting inner pressure of a balloon according to the first embodiment;

FIG. 6 is a schematic configuration diagram of the balloon control device according to the second embodiment;

FIG. 7 is a flowchart illustrating a method for detecting inner pressure of a balloon according to the second embodiment;

FIG. 8 is a schematic configuration diagram of a balloon control device according to the third embodiment;

FIG. 9 is a flowchart illustrating a method for detecting an inner pressure of a balloon according to the third embodiment;

FIG. 10 is a schematic configuration diagram of a balloon control device according to the fourth embodiment;

FIG. 11 is a flowchart illustrating a method for detecting an inner pressure of a balloon according to the fourth embodiment;

FIG. 12 is a system configuration diagram illustrating an embodiment of an endoscope apparatus provided with a supplementary tool for an endoscope; and

FIG. 13 is a system configuration diagram illustrating an embodiment of an endoscope apparatus including a treatment tool.

### DETAILED DESCRIPTION OF THE PREFERRED EMBODIMENTS

Hereinafter, embodiments of the presently disclosed subject matter will be described in detail with reference to the attached drawings.

### [Description of Endoscope Apparatus]

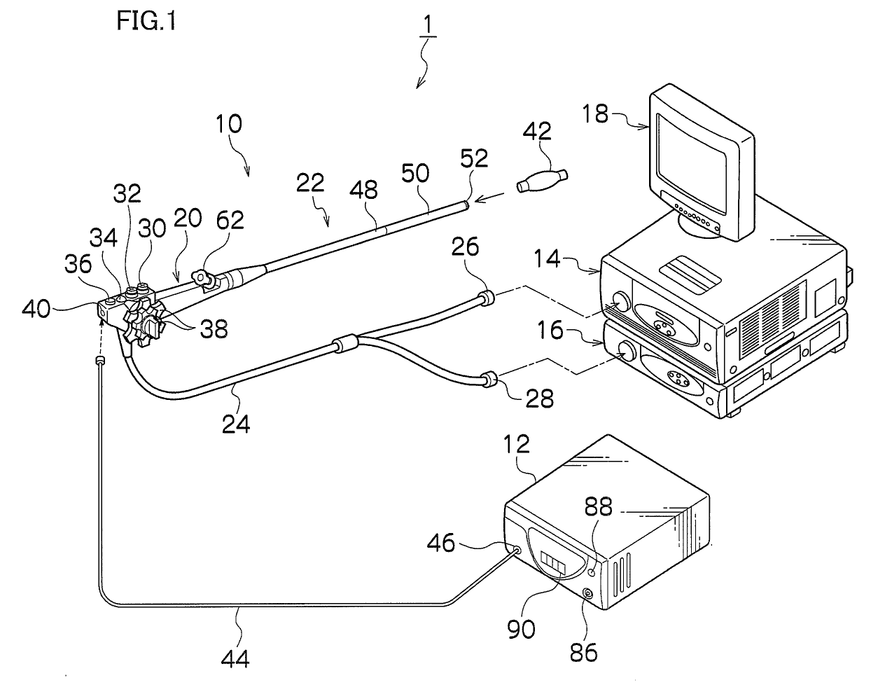

FIG. 1 is a system configuration diagram illustrating an embodiment of an endoscope apparatus 1 including an inner pressure detection device for an inflation and deflation member according to the presently disclosed subject matter.

As illustrated in FIG. 1, the endoscope apparatus 1 includes an endoscope 10, balloon control devices 12, a light source device 14, a processor 16 and a monitor 18. The inner pressure detection device for the inflation and deflation member according to the presently disclosed subject matter is included in each of the balloon control devices 12A to 12D according to the first to fourth embodiments as will be described later.

The endoscope 10 includes an operation part 20 which can be on hand of an operator, and configured to receive an instruction of an operator and an insertion portion 22 which is connected to the operation part 20 and is inserted into a body cavity. A universal cable 24 is connected to the operation part 20, and a light source connector 26 and an electrical connector 28 are provided at a distal end of the universal cable 24. The light source connector 26 is attachably and detachably connected to the light source device 14, and thereby, an illumination light is sent to an illumination optical system (not illustrated) provided at a distal end of the insertion portion 22. Further, the electrical connector 28 is attachably and detachably connected to the processor 16.

On the operation part 20, an air supply/water supply button 30, a suction button 32, a shutter button 34 and a function switching button 36 are arranged, and a pair of angle knobs 38 and 38 are provided. A supply/exhaust tube port 40 is provided at a proximal end portion of the operation part 20.

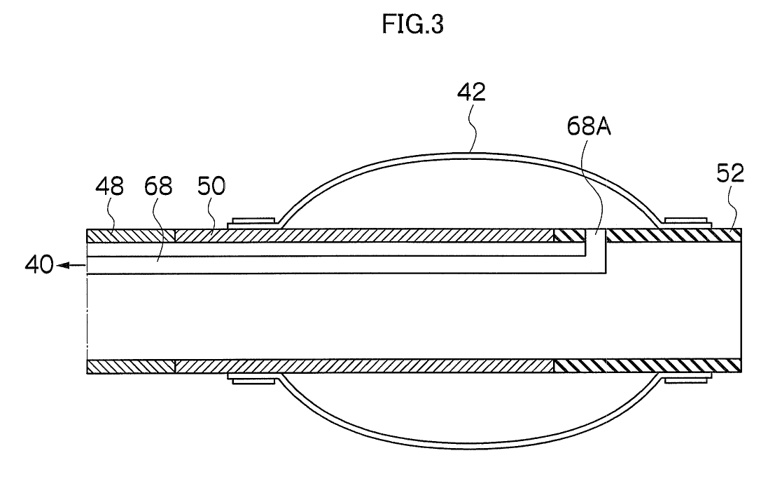

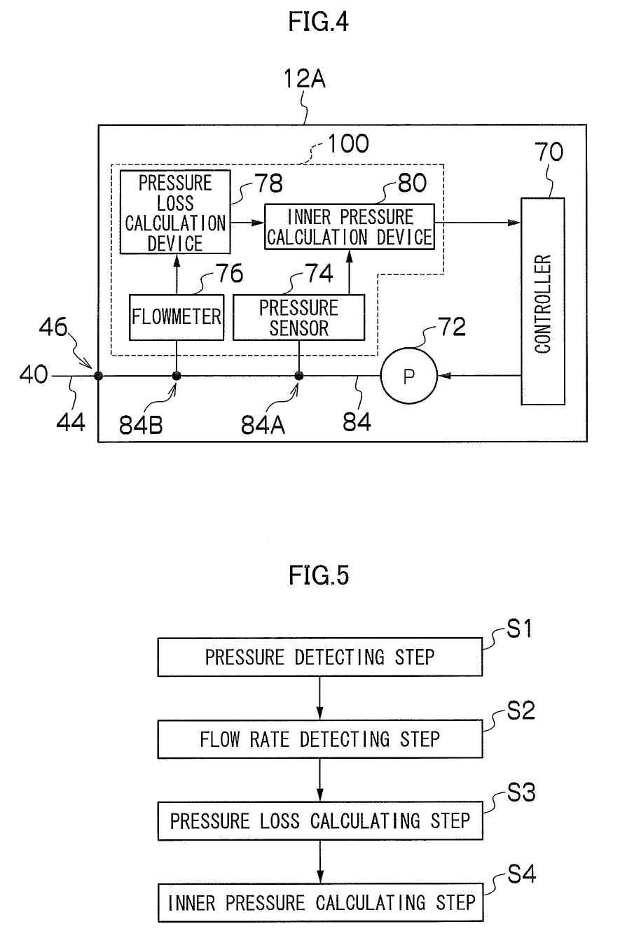

The supply/exhaust tube port 40 communicates with an inside of a balloon 42 through a supply/exhaust tube 68 included in the insertion portion 22, which will be described later (see FIG. 3). One end side of a tube 44 is connected to the supply/exhaust tube port 40, and another end side of the tube 44 is connected to a connecting portion 46 provided on a front surface of the balloon control device 12. The tube 44 functions as a supply/exhaust tube for supplying and exhausting a fluid to the inside of the balloon 42. Thereby, a pump 72 (see FIGS. 4, 6, 8 and 10) communicates with the inside of the balloon 42 through a supply/exhaust tube 84 included in the balloon control device 12, which will be described later.

A fluid is supplied or exhausted from the supply/exhaust tube port 40 by the pump 72, and thereby, the balloon 42 which will be described later can be inflated or deflated. As a fluid to be used, air, inert gas, water or the like can be properly selected.

The insertion portion 22 includes a flexible portion 48, a bending portion 50 and a distal end portion 52. The flexible portion 48, the bending portion 50 and the distal end portion 52 are arranged in sequence from the operation part 20 side. The flexible portion 48 has sufficient flexibility, and is connected to a proximal end side of the bending portion 50.

The bending portion 50 is remotely controlled to bend by rotating the angle knobs 38 and 38 of the operation part 20. For example, in the bending portion 50, a plurality of cylindrical node rings (guide rings) are rotatably connected with a guide pin, and a plurality of operation wires are inserted through the inside of the node rings to cause the guide pin to guide the operation wires. By pushing and pulling the operation wires by rotating the angle knobs 38 and 38, the node rings are rotated so as to bend the bending portion 50. By bending the bending portion 50, the distal end portion 52 can be directed to a desired direction.

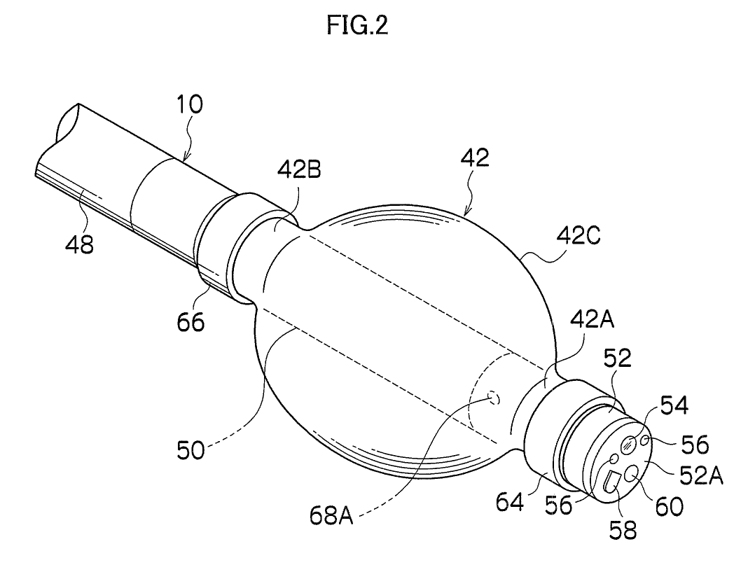

FIG. 2 is an enlarged perspective view of the distal end portion 52. As illustrated in FIG. 2, a distal end surface 52A of the distal end portion 52 is provided with an observation optical system 54, illumination optical systems 56 and 56, an air supply/water supply nozzle 58 and a forceps port 60.

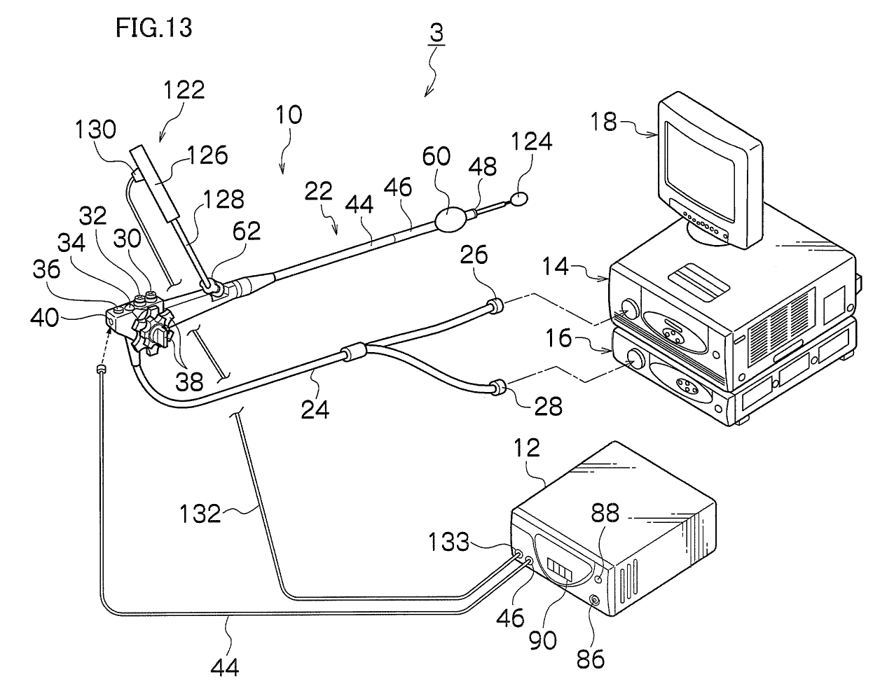

The forceps port 60 communicates with a forceps insertion portion 62 illustrated in FIG. 1. Therefore, by inserting a treatment tool such as forceps or an endoscope insertion guide tool from the forceps insertion portion 62, the treatment tool can be led out from the forceps port 60. As the details will be described later, an endoscope insertion guide tool 122 (see FIG. 13) includes a balloon 124, and is for guiding the insertion portion 22 of the endoscope 10.

Incidentally, as illustrated in FIG. 2, the balloon 42 formed by an elastic body such as rubber is fitted on an outer periphery surface of the insertion portion 22. The balloon 42 is formed into a substantially cylindrical shape constricted at its end portions, and includes a distal end portion 42A and a proximal end portion 42B, and a central swelling portion 42C. The diameters of distal end portion 42A and proximal end portion 42B are smaller than the central swelling portion 42C. The insertion portion 22 is inserted through the balloon 42 and is disposed at a predetermined position of the insertion portion 22, and thereafter, rubber rings 64 and 66 are fitted onto the distal end portion 42A and the proximal end portion 42B respectively. Then, the balloon 42 is fixed to the insertion portion 22. As illustrated in FIG. 2, the balloon 42 is disposed over the area from the distal end portion 52 to the bending portion 50 in this case.

The method for fixing the distal end portion 42A and the proximal end portion 42B is not especially limited, and they may be fixed by winding a thread around them. Further, the shape of the balloon 42 is not limited to the substantially cylindrical shape constricted at the end portions thereof, but may be formed into a spherical shape or the like.

FIG. 3 is a sectional view of the distal end of the insertion portion 22 and vicinity thereof. As illustrated in FIG. 3, a supply/exhaust tube 68 is formed inside the insertion portion 22. The proximal end of the supply/exhaust tube 68 communicates with the supply/exhaust tube port 40 (see FIG. 1) to supply and exhaust a fluid of the inside of the balloon 42. The distal end side of the supply/exhaust tube 68 communicates with the balloon 42 via an opening 68A which is formed in the outer peripheral surface of the distal end portion 52 and is included in the inside of the balloon 42.

When operating the endoscope apparatus 1, the insertion portion 22 is inserted by a push method (i.e. the insertion portion 22 is pushed into the body), and is fixed to the inside of the body (for example, a large intestine). In order to fix the insertion portion 22 to the inside of a body, the balloon 42 can be inflated, if necessary. Subsequently, the insertion portion 22 is pulled, and the shape of the inside of the body (for example, the tube geometry of the inside of the large intestine) is simplified. Then, the balloon 42 is deflated, and the insertion portion 22 is further inserted into the deep part of the intestinal canal.

For example, the insertion portion 22 is inserted from the anus of a subject. When the distal end of the insertion portion 22 passes the colon sigmoideum, the balloon 42 is inflated to fix the insertion portion 22 to the intestinal canal. Next, the insertion portion 22 is pulled to make the colon sigmoideum substantially rectilinear (i.e. the shape of the colon sigmoideum is simplified). Next, the balloon 42 is deflated, and the distal end of the insertion portion 22 is inserted into the deeper part of the intestinal canal. Thereby, the insertion portion 22 can be inserted into the deep part of the intestinal canal.

Here, when the balloon 42 is to be inflated or deflated, the internal balloon 42 is controlled while being detected by the balloon control device 12.

Next, the balloon control device 12 included in the inner pressure detection device for inflation and deflation member according to the presently disclosed subject matter will be described.

### First Embodiment

FIG. 4 is a schematic configuration diagram of the balloon control device 12A included in the inner pressure detection device of the first embodiment. In the first embodiment, the balloon control device 12A includes a controller 70, a pump 72, a pressure sensor 74 as a pressure detection device, a flowmeter 76 as a flow rate detection device, a pressure loss calculation device 78 and an inner pressure calculation device 80. The pressure sensor 74, the flowmeter 76, the pressure loss calculation device 78 and the inner pressure calculation device 80 correspond to the inner pressure detection device 100 for the inflation and deflation member in the present embodiment.

The pump 72 is connected to the supply/exhaust tube port 40 (see FIG. 1) through the tube 44, the connecting portion 46 and the supply/exhaust tube 84.

A power supply switch 86, a stop switch 88 and a pressure display section 90 are provided on the front surface of the balloon control device 12A (see FIG. 1). The pressure display section 90 is a panel (display panel) configured to display the pressure value in the balloon 42. An error code is displayed on the pressure display section 90 at the time of occurrence of abnormality such as a balloon break.

The balloon control device 12A can control the pump 72 with the controller 70, supply the fluid to the balloon 42 to inflate the balloon, and exhaust the fluid from the balloon 42 to deflate the balloon. At this time, the balloon control device 12A detects the inner pressure of the balloon 42 by the inner pressure detection device 100, and controls the pump 72 by the controller 70 based on the detection result to control the inner pressure of the balloon 42.

In the present embodiment, the inner pressure of the balloon 42 is detected by the inner pressure detection device 100 as follows. FIG. 5 is a flowchart illustrating a method for detecting the inner pressure of the balloon 42 according to the first embodiment.

First, the pressure of the fluid at a detection position 84A in the supply/exhaust tube 84 is detected by the pressure sensor 74 (pressure detecting step of step S1). Information of the pressure value detected by the pressure sensor 74 is sent to the inner pressure calculation device 80. The detection position 84A is provided between the connecting portion 46 and the pump 72. Further, the flowmeter 76 detects the flow rate of the fluid at a detection position 84B in the supply/exhaust tube 84 (flow rate detecting step of step S2). The detection position by the flowmeter 76 is not limited to the detection position 84B, but may be between the detection position 84A and the pump 72 in the supply/exhaust tube 84.

Next, based on the flow rate detected by the flowmeter 76, the pressure loss of the fluid between the position of the pressure sensor 74 and the position of the balloon 42 is calculated by the pressure loss calculation device 78 (pressure loss calculating step of step S3). More specifically, the pressure loss calculation device 78 calculates the pressure loss of the fluid in the supply/exhaust tube 84 in the balloon control device 12, the tube 44 and the supply/exhaust tube 68 in the insertion portion 22 between the detection position 84A of the pressure sensor 74 and the opening 68A of the supply/exhaust tube 68.

Here, the pressure loss can be calculated by a method using a well-known theoretical formula concerning the pressure loss in the tube (conduit line). The theoretical formula can be derived based on the hydrodynamics such as the Hagen-Poiseuille's law.

Further, the pressure loss can be calculated by a method using a table which defines the relationship between the flow rate of the fluid and the pressure loss in the tube (supply/exhaust tube). In the case of the method, information concerning the table which defines the relationship between the flow rate of the fluid and the pressure loss in the supply/exhaust tube 84, the tube 44 and the supply/exhaust tube 68 is stored in the pressure loss calculation device 78 in advance.

The pressure loss calculation device 78 can select the method for calculating these pressure losses based on, for example, the pressure accuracy required when controlling the inner pressure of the balloon 42. The pressure loss calculation device 78 can select the method for calculating the pressure loss automatically. The pressure loss calculation device 78 can select the method for calculating the pressure loss based on an instruction inputted by the operator through a man/machine interface device (not illustrated) included in the balloon control device 12A such as an operation panel at the balloon control device 12A.

Next, the inner pressure calculation device 80 calculates the inner pressure of the balloon 42 (inner pressure calculating step of step S4). For example, the inner pressure calculation device 80 adds the value of the pressure loss calculated by the pressure loss calculation device 78 to the pressure value detected by the pressure sensor 74, or subtracts the value of the calculated pressure loss from the detected pressure value to calculate the inner pressure of the balloon 42.

More specifically, when the fluid is supplied to the balloon 42, the value of the pressure loss calculated by the pressure loss calculation device 78 is subtracted from the pressure value detected by the pressure sensor 74. When the fluid is exhausted from the balloon 42, the value of the pressure loss calculated by the pressure loss calculation device 78 is added to the pressure value detected by the pressure sensor 74. Then, the inner pressure of the balloon 42 is calculated. The information of the pressure value detected by the pressure sensor 74 is sent to the inner pressure calculation device 80.

As described above, according to the inner pressure detection device 100 of the balloon 42 of the present embodiment, the pressure loss in the tubes such as the supply/exhaust tube 84, the tube 44 and the supply/exhaust tube 68 between the detection position 84A of the pressure sensor 74 and the opening 68A of the insertion portion 22 is added or subtracted to or from the pressure value detected by the pressure sensor 74.

Therefore, the inner pressure of the balloon 42 which does not include the pressure loss can be accurately detected.

Accordingly, based on the detected inner pressure, the pump 72 can be driven with the maximum capacity by the controller 70 to supply or exhaust the fluid in the balloon 42 until the inner pressure of the balloon 42 reaches a predetermined pressure. Therefore, the balloon 42 can be inflated or deflated quickly and safely.

Further, according to the embodiment, the pressure sensor 74 can not be provided inside the insertion portion 22, and the insertion portion 22 can be made compact.

The balloon control device 12A may include a monitor (not illustrated) dedicated for a balloon which displays the inner pressure value and an inflated or deflated state (level) of the balloon 42 when the balloon 42 is inflated or deflated. Further, the inner pressure value and the inflated or deflated state of the balloon 42 may be displayed on the monitor 18. For example, the inner pressure value and the inflated or deflated state of the balloon 42 may be superimposed on the observation image which is imaged by the endoscope 10, and is displayed on the monitor 18.

### Second Embodiment

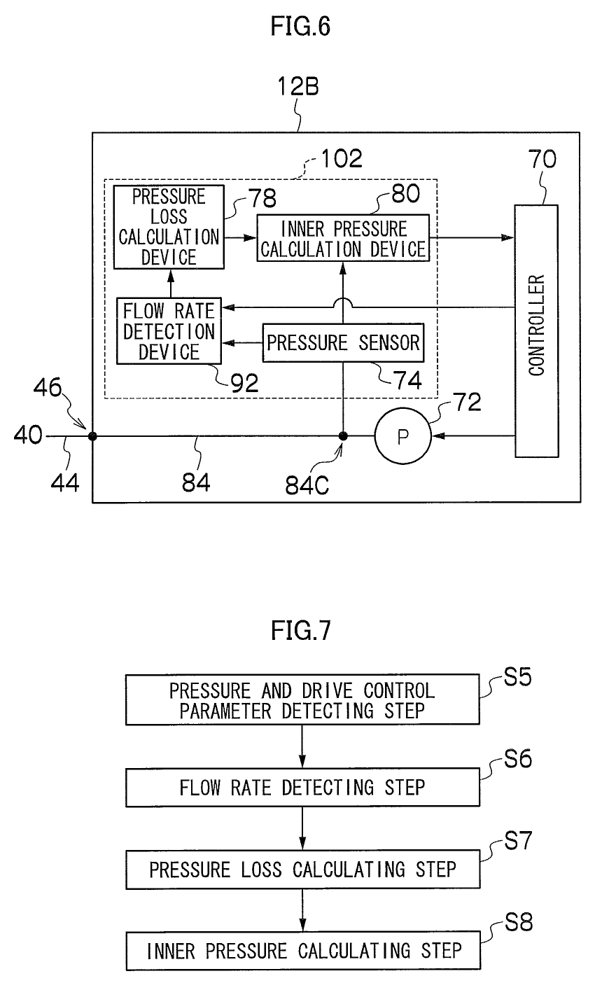

FIG. 6 is a schematic configuration diagram of a balloon control device 12B included in an inner pressure detection device according to the second embodiment. In the second embodiment, the balloon control device 12B includes a flow rate detection device 92 in place of the flowmeter 76 of the first embodiment. Further, especially in the second embodiment, the pressure sensor 74 is provided in the vicinity of the pump 72. And, information concerning the pressure flow rate characteristic of the pump 72 is stored in the flow rate detection device 92 in advance. Here, the pressure flow rate characteristic defines a relationship of a drive control parameter (a parameter relating to the pump 72; for example, voltage applied to the pump 72, frequency (drive frequency) applied to the pump 72), the discharge pressure of the fluid discharged by the pump 72 and the flow rate of the fluid. The pressure flow rate characteristic is determined in accordance with the kind (model) of the pump or the like.

The other components of the second embodiment are similar to the corresponding components of the first embodiment respectively. An inner pressure detection device 102 for an inflation and deflation member of the present embodiment includes the pressure sensor 74, the pressure loss calculation device 78, the inner pressure calculation device 80 and the flow rate detection device 92.

In the present embodiment, the inner pressure of the balloon 42 is detected by the inner pressure detection device 102 as follows. FIG. 7 is a flowchart illustrating a method for detecting the inner pressure of the balloon 42 according to the second embodiment.

First, the pressure of the fluid at a detection position 84C located in the vicinity of the pump 72 in the supply/exhaust tube 84 is detected by the pressure sensor 74. The supply/exhaust tube 84 is between the connecting portion 46 and the pump 72. Also, the drive control parameter by which the controller 70 performs drive control of the pump 72 is detected (pressure and drive control parameter detecting step of step S5). Information of the pressure value detected by the pressure sensor 74 is sent to the inner pressure calculation device 80. Next, based on the pressure detected by the pressure sensor 74, and the drive control parameter, the flow rate of the fluid in the supply/exhaust tube 84 is detected by using the information of the pressure flow rate characteristic of the pump 72 which is stored in advance, by the flow rate detection device 92 (flow rate detecting step of Step S6).

Next, based on the flow rate detected by the flow rate detection device 92, the pressure loss of the fluid between the position of the pressure sensor 74 and the position of the balloon 42 is calculated by the pressure loss calculation device 78 (pressure loss calculating step of step S7). More specifically, the pressure loss calculation device 78 calculates the pressure loss of the fluid in the supply/exhaust tube 84, the tube 44 and the supply/exhaust tube 68 which are between the detection position 84C of the pressure sensor 74 and the opening 68A of the supply/exhaust tube 68 (see FIG. 3). The method for calculating the pressure loss in the pressure loss calculation device 78 is similar to that of the first embodiment.

Next, the inner pressure calculation device 80 calculates the inner pressure of the balloon 42 (inner pressure calculating step of step S8). For example, the inner pressure calculation device 80 adds the value of the pressure loss calculated by the pressure loss calculation device 78 to the pressure value detected by the pressure sensor 74, or subtracts the value of the calculated pressure loss from the detected pressure value to calculate the inner pressure of the balloon 42.

As above, according to the inner pressure detection device 102 of the balloon 42 of the present embodiment, the inner pressure of the balloon 42 also can be detected accurately, and inflation or deflation of the balloon 42 can be performed quickly and safely. Further, the insertion portion 22 can be made compact.

Further, according to the present embodiment, the flowmeter 76 is not provided, and therefore, the configuration can be made simpler than the first embodiment.

### Third Embodiment

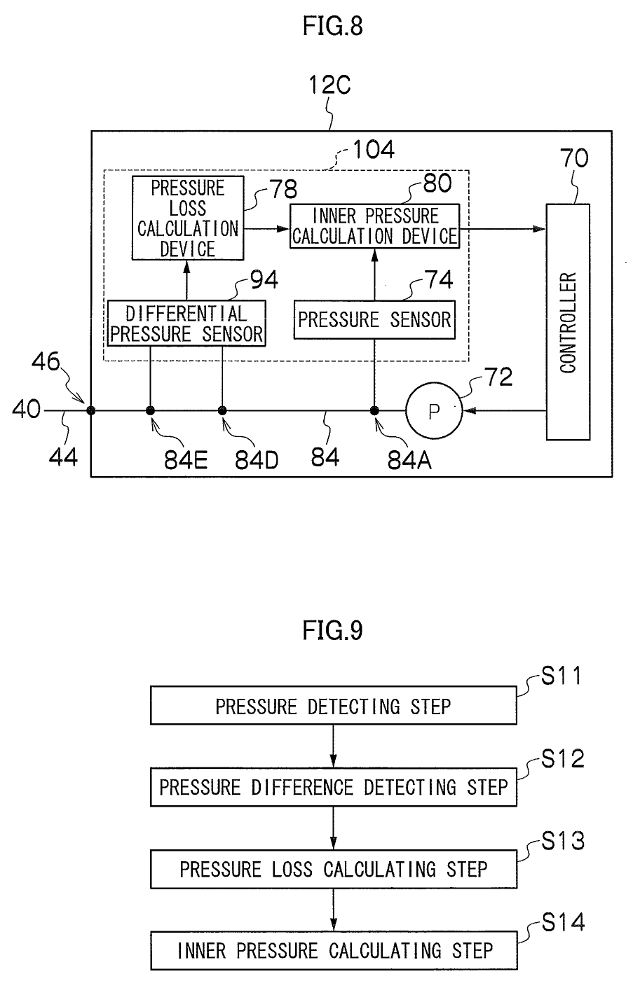

FIG. 8 is a schematic configuration diagram of the balloon control device 12C included in an inner pressure detection device according to the third embodiment. In the third embodiment, the balloon control device 12C includes a differential pressure sensor 94 as a pressure difference detection device in place of the flowmeter 76 and the flow rate detection device 92 of the first and second embodiments. The other components of the third embodiment are similar to the corresponding components of the first and second embodiments respectively. An inner pressure detection device 104 for an inflation and deflation member of the present embodiment includes the pressure sensor 74, the pressure loss calculation device 78, the inner pressure calculation device 80 and the differential pressure sensor 94.

In the present embodiment, the inner pressure of the balloon 42 is detected by the inner pressure detection device 104 as follows. FIG. 9 is a flowchart illustrating a method for detecting the inner pressure of the balloon 42 according to the third embodiment.

First, the pressure of the fluid at the detection position 84A in the supply/exhaust tube 84 is detected by the pressure sensor 74 (pressure detecting step of step S11). The information of the pressure value detected by the pressure sensor 74 is sent to the inner pressure calculation device 80. The supply/exhaust tube 84 is provided between the connecting portion 46 and the pump 72. Further, the pressure difference of the fluid between a detection position 84D and a detection position 84E in the supply/exhaust tube 84 is detected by the differential pressure sensor 94 (pressure difference detecting step of step S12).

Next, based on the pressure difference detected by the differential pressure sensor 94, the pressure loss of the fluid between the position of the pressure sensor 74 and the position of the balloon 42 is calculated by the pressure loss calculation device 78 (pressure loss calculating step of step S13). More specifically, the pressure loss of the fluid in the supply/exhaust tube 84, the tube 44 and the supply/exhaust tube 68 which are located between the detection position 84A of the pressure sensor 74 and the opening 68A of the supply/exhaust tube 68 (see FIG. 3) is calculated.

More specifically, the pressure loss is calculated as follows. In the following Expression 1, L1 represents the length along the supply/exhaust tube 84, the tube 44 and the supply/exhaust tube 68 between the detection position 84A and the opening 68A, and L2 represents the length along the supply/exhaust tube 84 between the detection position 84D and the detection position 84E. Further, PL1 represents the pressure loss between the detection position 84A and the opening 68A, and PL2 represents the pressure difference detected by the differential pressure sensor 94.

The pressure loss PL1 is calculated by using the following expression.

PL1=PL2×(L1/L2) [Expression 1]

The method for calculating the pressure loss PL1 is not limited to the method using the Expression 1 based on the detected pressure difference PL2. The pressure loss PL1 may be drawn from a table which defines the relationship of the pressure loss PL1 (the pressure loss PL1 in the table can be calculated by Expression 1) and the pressure difference PL2, and is stored in the pressure loss calculation device 78 in advance.

Next, the inner pressure calculation device 80 calculates the inner pressure of the balloon 42 (inner pressure calculating step of step S14). For example, the inner pressure calculation device 80 adds the value of the pressure loss PL1 calculated by the pressure loss calculation device 78 to the pressure value detected by the pressure sensor 74, or subtracts the value of the calculated pressure loss PL1 from the detected pressure value to calculate the inner pressure of the balloon 42.

As above, according to the inner pressure detection device 104 of the balloon 42 of the third embodiment, the inner pressure of the balloon 42 can be detected accurately, and inflation or deflation of the balloon 42 can be performed quickly and safely. Further, the insertion portion 22 can be made compact.

### Fourth Embodiment

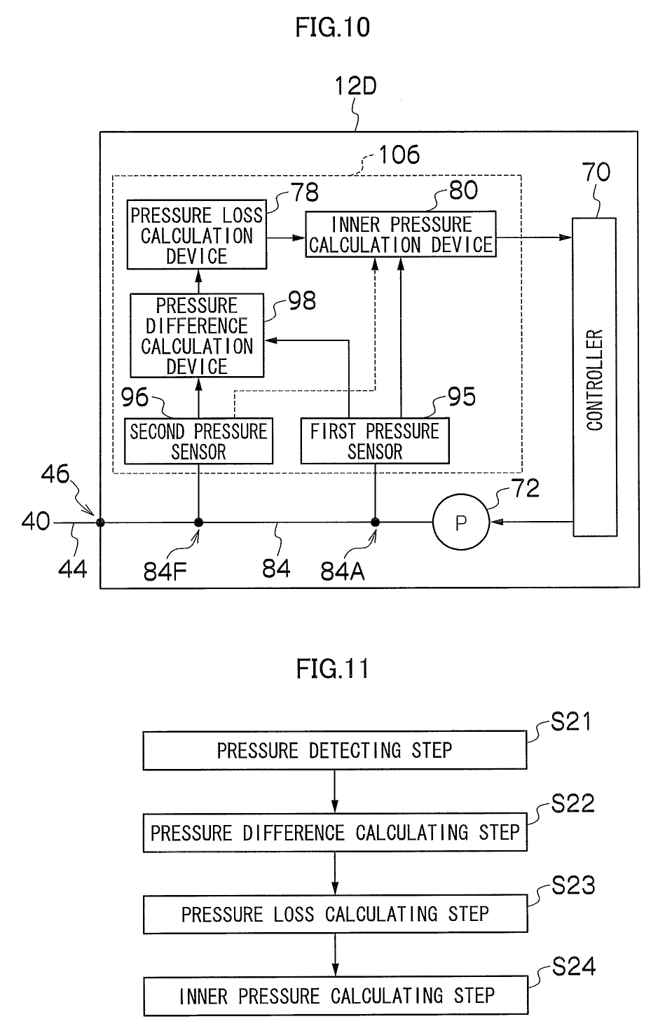

FIG. 10 is a schematic configuration diagram of the balloon control device 12D included in an inner pressure detection device according to the fourth embodiment. In the fourth embodiment, the balloon control device 12D includes a second pressure sensor 96 in place of the differential pressure sensor 94 of the third embodiment, and further includes a pressure difference calculation device 98. The other components of the fourth embodiment are similar to the corresponding components of the third embodiment.

In the present embodiment, pressure sensors as pressure detection devices are provided at two spots in the supply/exhaust tube 84. As illustrated in FIG. 10, the balloon control device 12D includes the first pressure sensor 95 and the second pressure sensor 96. The first pressure sensor 95 corresponds to the pressure sensors 74 of the first to third embodiments. As illustrated in FIG. 10, the second pressure sensor 96 is provided at a position closer to the connecting portion 46 (closer to the balloon 42) than the first pressure sensor 95.

The inner pressure detection device 106 for the inflation and deflation member of the present embodiment includes the pressure loss calculation device 78, the inner pressure calculation device 80, the first pressure sensor 95, the second pressure sensor 96 and the pressure difference calculation device 98.

In the present embodiment, the inner pressure of the balloon 42 is detected by the inner pressure detection device 106 as follows. FIG. 11 is a flowchart illustrating a method for detecting the inner pressure of the balloon 42 according to the fourth embodiment.

First, the pressure value of the fluid at the detection position 84A in the supply/exhaust tube 84 provided between the connecting portion 46 and the pump 72 is detected by the first pressure sensor 95. And, (at the same time) the pressure value of the fluid at the detection position 84F in the supply/exhaust tube 84 is detected by the second pressure sensor 96 (pressure detecting step of step S21). Information of the pressure value detected by the first pressure sensor 95 is sent to the inner pressure calculation device 80.

Next, based on the pressure values detected by the first pressure sensor 95 and the second pressure sensor 96, the pressure difference of the fluid between the detection position 84A and the detection position 84F is calculated by the pressure difference calculation device 98 (pressure difference calculating step of step S22).

Next, based on the pressure difference calculated by the pressure difference calculation device 98, the pressure loss of the fluid between the position of the first pressure sensor 95 and the position of the balloon 42 is calculated by the pressure loss calculation device 78 (pressure loss calculating step of step S23). More specifically, the pressure loss of the fluid in the supply/exhaust tube 84, the tube 44 and the supply/exhaust tube 68 which are located between the detection position 84A of the first pressure sensor 95 and the opening 68A of the supply/exhaust tube 68 (see FIG. 3) is calculated.

More specifically, the pressure loss PL1 is calculated by using Expression 2 described below.

PL1=PL3×(L1/L3) [Expression 2]

In the Expression 2, L1 represents the length along the supply/exhaust tube 84, the tube 44 and the supply/exhaust tube 68 between the detection position 84A to the opening 68A, and L3 represents the length along the supply/exhaust tube 84 between the detection position 84A and the detection position 84F. Further, PL1 represents the pressure loss between the detection position 84A and the opening 68A, and PL3 represents the pressure difference between the detection position 84A and the detection position 84F calculated by the pressure difference calculation device 98.

Next, the inner pressure calculation device 80 calculates the inner pressure of the balloon 42 (inner pressure calculating step of step S24). For example, the inner pressure calculation device 80 adds the value of the pressure loss PL1 calculated by the pressure loss calculation device 78 to the pressure value detected by the pressure sensor 74, or subtracts the value of the calculated pressure loss PL1 from the detected pressure value to calculate the inner pressure of the balloon 42.

Further, instead of the pressure loss between the detection position 84A of the first pressure sensor 95 and the opening 68A of the supply/exhaust tube 68, the pressure loss between the detection position 84F of the second pressure sensor 96 and the opening 68A of the supply/exhaust tube 68 may be calculated. In this case, on use of the Expression 2, the length between the detection position 84F and the opening 68A is set as L1 and the pressure loss between the detection position 84F and the opening 68A is set as PL1.

When the pressure loss between the detection position 84F of the second pressure sensor 96 and the opening 68A of the supply/exhaust tube 68 is calculated by the pressure loss calculation device 78, the inner pressure calculation device 80 adds the value of the pressure loss PL1 to the pressure value detected by the second pressure sensor 96, or subtracted the value of the pressure loss PL1 from the pressure value detected by the second pressure sensor 96 to calculate the inner pressure of the balloon 42. In this case, as illustrated by the dotted line arrow of FIG. 10, information of the pressure value detected by the second pressure sensor 96 is sent to the inner pressure calculation device 80.

As above, according to the inner pressure detection device 106 of the balloon 42 of the fourth embodiment, the inner pressure of the balloon 42 also can be detected accurately, and inflation or deflation of the balloon 42 can be performed quickly and safely. Further, the insertion portion 22 can be made compact.

### [Supplementary Tool for an Endoscope]

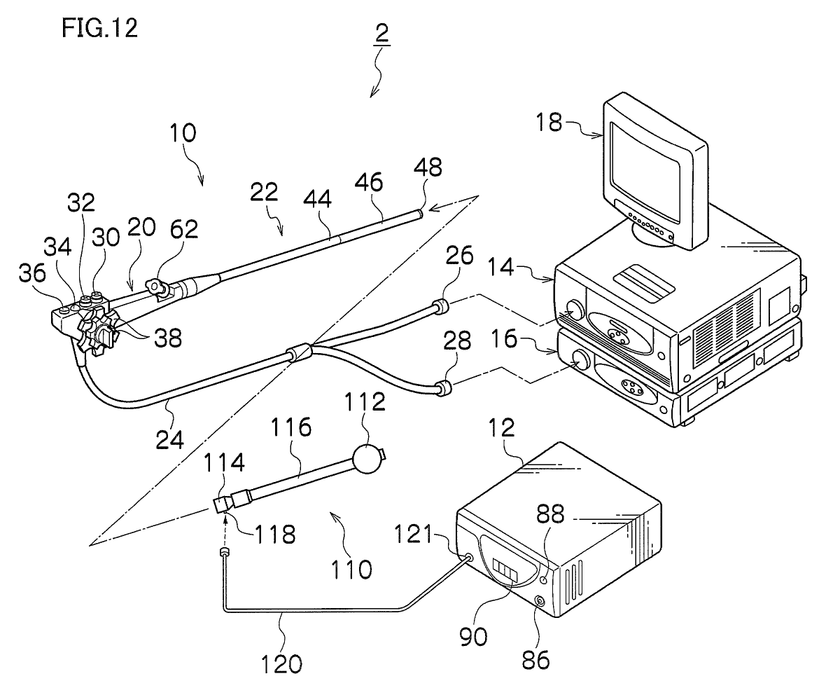

In the above description, application to the balloon 42 in the insertion portion 22 of the endoscope apparatus 1 is considered, but other than this, application to the balloon provided at a supplementary tool for an endoscope as follows as an intracavital insertion portion is also conceivable. FIG. 12 is a system configuration diagram illustrating an embodiment of an endoscope apparatus 2 provided with a supplementary tool (insertion auxiliary tool) 110 for the endoscope.

In the endoscope apparatus 2 illustrated in FIG. 12, the inner pressure of a balloon 112 provided at the insertion auxiliary tool 110 can be accurately detected by using the balloon control devices 12A to 12D including the inner pressure detection devices 100, 102, 104 and 106 of the first to fourth embodiments. Therefore, inflation or deflation of the balloon 112 can be performed quickly and safely. Further, a pressure sensor does not have to be provided inside the insertion auxiliary tool 110, and the insertion auxiliary tool 110 can be made compact.

Here, a configuration of the endoscope apparatus 2 will be described.

As illustrated in FIG. 12, the insertion portion 22 of the endoscope 10 is inserted through the insertion auxiliary tool 110. The insertion auxiliary tool 110 includes a grip portion 114 and a tube main body 116. The grip portion 114 is a portion for an operator to grip. The grip portion 114 is formed into a cylindrical shape by a hard material such as plastic, and a connector 118 is provided at a cylindrical portion. The tube main body 116 is fitted onto a distal end side of the grip portion 114, and fixed to the distal end side of the grip portion 114.

The tube main body 116 is formed into a substantially cylindrical shape by a flexible material such as polyurethane. The tube main body 116 is provided with the balloon 112 at a distal end side thereof, and the distal end side is opposite from a side to which the grip portion 114 is fixed.

A supply/exhaust tube (not illustrated) is provided in the insertion auxiliary tool 110. A distal end side of the supply/exhaust tube in the insertion auxiliary tool 110 communicates with the balloon 112. A proximal end side of the supply/exhaust tube in the insertion auxiliary tool 110 communicates with the connector 118. A tube 120 is connected to the connector 118. The supply/exhaust tube in the insertion auxiliary tool 110 is connected to the supply/exhaust tube 84 (see FIGS. 4, 6, 8 and 10) in the control device in the balloon control devices 12A to 12D via a connecting portion 121. The tube 120 can be used as the supply/exhaust tube for supplying and exhausting a fluid.

Further, the insertion portion 22 of the endoscope 10 may be provided with the balloon 42, and any one of the inner pressure detection devices 100, 102, 104 and 106 of the first to fourth embodiments may be separately included in order to detect the inner pressure of the balloon 42.

### [Treatment Tool for an Endoscope]

Further, application to the balloon provided at an endoscope treatment tool as follows as an intracavital insertion portion is also conceivable.

FIG. 13 is a system configuration diagram illustrating an embodiment of an endoscope apparatus 3 provided with an insertion guide tool 122 as an endoscope treatment tool.

In the endoscope apparatus 3 illustrated in FIG. 13, the inner pressure of a balloon 124 provided at the insertion guide tool 122 also can be accurately detected by the balloon control devices 12A to 12D including the inner pressure detection devices 100, 102, 104 and 106 according to the first to fourth embodiments. Therefore, inflation or deflation of the balloon 124 can be performed quickly and safely. Further, a pressure sensor does not have to be provided inside the insertion guide tool 122, and the insertion guide tool 122 can be made compact.

Here, a configuration of the endoscope apparatus 3 will be described.

As illustrated in FIG. 13, the insertion guide tool 122 is inserted from the forceps insertion portion 62, and is led out from the forceps port 60 (see FIG. 2) at the distal end portion 52. The insertion guide tool 122 includes a grip portion 126, and a linear member 128 connectively provided at the grip portion 126. The grip portion 126 can be a hard portion.

The linear member 128 is formed into a linear shape and has sufficient flexibility. A proximal end side of the linear member 128 is fixed to the grip portion 126. The balloon 124 is fitted onto an outer peripheral surface of a distal end of the linear member 128.

A supply/exhaust tube in the insertion guide tool 122 (not illustrated) is provided in the linear member 128. A distal end side of the supply/exhaust tube in the insertion guide tool 122 communicates with the balloon 124. A proximal end side of the supply/exhaust tube in the insertion guide tool 122 communicates with a connector 130. A tube 132 is connected to the connector 130. The supply/exhaust tube in the insertion guide tool 122 is connected to the supply/exhaust tube 84 in the balloon control devices 12A to 12D (see FIGS. 4, 6, 8 and 10) via a connecting portion 133. The tube 132 can be used as a supply/exhaust tube for supplying and exhausting a fluid.

Instead of providing the balloon 42 at the insertion portion 22, the insertion auxiliary tool 110 equipped with the balloon 112 in the above described endoscope apparatus 2 may be provided. In order to detect the inner pressure of the balloon 112, any one of the inner pressure detection devices 100, 102, 104 and 106 of the first to fourth embodiments may be included separately.

In the abovementioned description, although the inner pressure detection devices 100, 102, 104 and 106 is included in the balloon control devices 12A to 12D, the presently disclosed subject matter is not limited to this. For example, the inner pressure detection devices 100, 102, 104 and 106 may be separately configured outside of the balloon control devices 12A to 12D.

Further, in the abovementioned description, although the pressure sensor 74, the flowmeter 76, the differential pressure sensor 94, the first pressure sensor 95 and the second pressure sensor 96 are provided in the supply/exhaust tube 84, the presently disclosed subject matter is not limited to this. For example, they may be provided in the tubes 44, 120 and 132, the supply/exhaust tube 68, the supply/exhaust tube in the insertion auxiliary tool 110 or the supply/exhaust tube in the insertion guide tool 122.

The inner pressure detection device and the inner pressure detection method for the inflation and deflation member and the endoscope apparatus of the presently disclosed subject matter according to the presently disclosed subject matter is not limited to the above embodiments, and various improvements and modifications may be made within the range without departing from the gist of the presently disclosed subject matter as a matter of course.

What is claimed is:

1. An inner pressure detection device for an inflation and deflation member, comprising:
a pressure detection device which detects a pressure of a fluid at a first position in a proximal end side of a tube for supplying a fluid to an inflation and deflation member and exhausting a fluid from the inflation and deflation member, the inflation and deflation member included in an insertion portion configured to be inserted in a body cavity;
a pressure loss calculation device which calculates a pressure loss of the fluid in the tube between the first position and a position in a distal end side of the tube where the inflation and deflation member is provided; and
an inner pressure calculation device which calculates an inner pressure of the inflation and deflation member based on the pressure loss calculated by the pressure loss calculation device and the pressure detected by the pressure detection device.

2. The inner pressure detection device for an inflation and deflation member according to claim 1, further comprising:
a flow rate detection device which detects a flow rate of the fluid in the tube,
wherein the pressure loss calculation device calculates the pressure loss based on the flow rate detected by the flow rate detection device.

3. The inner pressure detection device for an inflation and deflation member according to claim 2,
wherein the pressure loss calculation device calculates the pressure loss by using a table defining a relationship of the flow rate and the pressure loss.

4. The inner pressure detection device for an inflation and deflation member according to claim 2,
wherein the pressure detection device is provided in a vicinity of a supply and exhaust device which supplies and exhausts the fluid in the tube, and
the flow rate detection device detects the flow rate based on the inner pressure detected by the pressure detection device by using information of a pressure flow rate characteristic of the supply and exhaust device, the flow rate characteristic defining a relationship of a drive control parameter of the supply and exhaust device, a pressure of the fluid supplied and exhausted by the supply and exhaust device and the flow rate of the fluid.

5. The inner pressure detection device for an inflation and deflation member according to claim 1, further comprising:
a pressure difference detection device which detects a pressure difference of the fluid between the first position and a second position in the tube,
wherein the pressure loss calculation device calculates the pressure loss based on the pressure difference detected by the pressure difference detection device.

6. The inner pressure detection device for an inflation and deflation member according to claim 1, further comprising:
a pressure difference calculation device,
wherein the pressure detection device detects pressure values at the first position and a second position in the tube,
the pressure difference calculation device calculates a pressure difference of the fluid between the first position and the second position based on the pressures detected by the pressure detection device, and
the pressure loss calculation device calculates the pressure loss based on the pressure difference calculated by the pressure difference calculation device.

7. The inner pressure detection device for an inflation and deflation member according to claim 5,
wherein the pressure loss calculation device calculates a pressure loss PL1 by using an expression defined as PL1=PL2×(L1/L2) when a length of the tube between the first position and a position where the inflation and deflation member is provided is set as L1, a length of the tube between the first position and the second position is set as L2, the pressure loss is set as PL1, and the pressure difference of the fluid between the first position and the second position is set as PL2.

8. The inner pressure detection device for an inflation and deflation member according to claim 6,
wherein the pressure loss calculation device calculates a pressure loss PL1 by using an expression defined as PL1=PL2×(L1/L2) when a length of the tube between the first position and a position where the inflation and deflation member is provided is set as L1, a length of the tube between the first position and the second position is set as L2, the pressure loss is set as PL1, and the pressure difference of the fluid between the first position and the second position is set as PL2.

9. The inner pressure detection device for an inflation and deflation member according to claim 1,
wherein the insertion portion is an insertion portion included in an endoscope.

10. The inner pressure detection device for an inflation and deflation member according to claim 1,
wherein the insertion portion is a supplementary tool for an endoscope through which an insertion portion included in an endoscope is inserted.

11. The inner pressure detection device for an inflation and deflation member according to claim 1,
wherein the insertion portion is an endoscope treatment tool which is led out from a forceps port of an endoscope.

12. An endoscope apparatus, comprising:
an insertion portion configured to be inserted into a body cavity;
an inflation and deflation member provided at the insertion portion;
a tube through which a fluid is supplied to and exhausted from an inside of the inflation and deflation member;
a supply and exhaust device which supplies and exhausts the fluid in the tube;
a pressure detection device which detects a pressure of the fluid at a first position in a proximal end side of the tube;
a pressure loss calculation device which calculates a pressure loss of the fluid in the tube between the first position and a position in a distal end side of the tube where the inflation and deflation member is provided; and
an inner pressure calculation device which calculates an inner pressure of the inflation and deflation member based on the pressure loss calculated by the pressure loss calculation device and the pressure detected by the pressure detection device.

13. An inner pressure detection method for an inflation and deflation member, comprising the step of:
detecting a pressure of a fluid at a first position in a proximal end side of a tube through which a fluid is supplied to and exhausted from an inside of an inflation and deflation member included in an insertion portion;
calculating a pressure loss of the fluid in the tube between the first position and a position in a distal end side of the tube where the inflation and deflation member is provided; and
calculating an inner pressure of the inflation and deflation member based on the calculated pressure loss and the detected pressure at the first position.
